(19) United States
(12) Patent Application Publication
(10) Pub. No.: US 2021/0093817 A1
(43) Pub. Date: Apr. 1, 2021

(54) DEVICES AND METHODS FOR INTRODUCING AN ENDOTRACHEAL TUBE

(71) Applicant: Through the Cords, LLC, Salt Lake City, UT (US)

(72) Inventors: Sean RUNNELS, Salt Lake City, UT (US); Wil ROBERGE, Salt Lake City, UT (US); Benjamin FOGG, Salt Lake City, UT (US)

(73) Assignee: Through the Cords, LLC, Salt Lake City, UT (US)

(21) Appl. No.: 15/733,362

(22) PCT Filed: Jan. 11, 2019
(86) PCT No.: PCT/US2019/013386

§ 371 Date: Jul. 9, 2020

Related U.S. Application Data
(60) Provisional application No. 62/616,427, filed on Jan. 12, 2018.

(51) Int. Cl. A61M 16/04; A61B 1/267

(52) U.S. Cl. CPC A61M 16/0488; A61B 1/267; A61M 2205/584; A61M 16/0434; A61M 16/0461

(57) ABSTRACT

An introducer usable with a tracheal intubation system is disclosed. Methods for using the introducer and systems that incorporate the introducer are also disclosed. In some examples, an introducer adapted for mounting an endotracheal tube comprises a handle that is configured to actuate a tip portion. The shaft comprises a distal shaft portion and a proximal shaft portion that have a continuous exterior surface. The proximal shaft portion has a body portion and the tip portion.

### BACKGROUND

Many surgical procedures are typically performed while the patient is under general anesthesia. During these procedures, the patient is given a combination of medications to cause a loss of consciousness and muscle paralysis. The medications that cause loss of consciousness and muscle paralysis also interfere with the patient's ability to breath. Accordingly, patients often undergo tracheal intubation during these procedures so that the patient may be connected to an external ventilator or breathing circuit. Patients may also be intubated for nonsurgical conditions in which enhanced oxygen delivery is required. Tracheal intubation may also be used in other circumstances.

During tracheal intubation, an endotracheal tube is placed in the patient's airway. Generally, the endotracheal tube is advanced through the patient's nose or mouth into the patient's trachea. The endotracheal tube is then connected to an external ventilator or breathing circuit. The ventilator is then able to breath for the patient, delivering oxygen into the patient's lungs.

The patient's vocal cords and the space between them form the entrance to the trachea, these structures are also known as the glottis. The glottis is visible from and may be accessed through the pharynx. The pharynx is the portion of the upper airway that is located behind the patient's mouth and below the patient's nasal cavity. The mouth and the nasal cavity meet in the pharynx. Additionally, the esophagus and the glottis may be accessed through the pharynx. During the intubation process, the endotracheal tube must be carefully advanced through the patient's pharynx and placed through the vocal cords into the trachea. In addition, it is critical that the endotracheal tube be placed at the proper depth once in the trachea. If it is placed to shallow in the trachea, it can fall out. If it is placed too deep, only one lung may be ventilated resulting in poor oxygen delivery to the blood or hyperventilation on the ventilated lung, and hypoventilation to the non-ventilated lung. All of this can result in patient injury or death.

The intubation process interferes with the patient's ability to breathe and thus deliver oxygen to the body independently. If the patient is without oxygen for more than two or three minutes, tissue injury may occur, which can lead to death or permanent brain damage. Accordingly, the intubation process must be performed quickly and accurately.

### SUMMARY

In general terms, this disclosure is directed to an introducer for use with a tracheal intubation system. In one possible configuration and by non-limiting example, the tracheal intubation system allows a medical professional to properly position an endotracheal tube in a normal or difficult airway quickly, accurately, and safely. In another configuration and by non-limiting example, the tracheal intubation system allows a medical professional to properly perform an endotracheal tube exchange procedure quickly, accurately, and safely. One aspect is an introducer for mounting an endotracheal tube, the introducer comprising: a shaft comprising: a proximal shaft portion; and a distal shaft portion comprising a distal tip portion extending from the distal shaft portion, the shaft including a plurality of depth assessment bands, each depth assessment band having a visually distinct color or pattern from an adjacent depth assessment band, and the distal tip portion having a rounded shape and a closed end; and a handle removeably connected to the proximal shaft portion.

Another aspect is a method for inserting an endotracheal tube in a patient comprising: inserting a blade of a laryngoscope in a mouth of the patient; viewing a trachea of the patient with the laryngoscope; inserting an introducer comprising a handle into a trachea of the patient; removing the handle from the introducer; inserting the endotracheal tube over the introducer and into the trachea of the patient; and removing the introducer from the endotracheal tube, while the endotracheal tube remains in the patient.

A further aspect is an introducer for mounting an endotracheal tube, the introducer comprising: a shaft comprising: a proximal shaft portion; a distal shaft portion comprising a distal tip portion extending from the shaft portion, the shaft comprises a plurality of qualitative depth assessment band, each depth assessment band having a visually distinct color or pattern from an adjacent depth assessment band, and a tip having a round shape and a closed end; and a control wire at least partially disposed within both the distal shaft portion and the proximal shaft portion and configured to cause the tip portion of the proximal shaft portion to maintain a curved configuration.

### DESCRIPTION OF THE DRAWINGS

FIG. 1 is a diagram of an example tracheal intubation system including a laryngoscope being used to intubate a patient.

FIG. 2 is a perspective view of an example laryngoscope.

FIG. 3 is a perspective view of an example introducer.

FIG. 4 is a perspective view of an example endotracheal tube.

FIG. 5 is a flowchart of an example process of placing an endotracheal tube in a patient using an example tracheal intubation system including a laryngoscope.

FIG. 6 is a cross-sectional view of a patient after a laryngoscope is positioned to view the glottis during an intubation procedure using an example tracheal intubation system including a laryngoscope.

FIG. 7 is a cross-sectional view of a patient after the tip of the introducer is advanced into the field of view of a laryngoscope during an intubation procedure using an example tracheal intubation system including a laryngoscope.

FIG. 8 is a cross-sectional view of a patient after the tip of an introducer is advanced into the trachea to a second depth-assessment band during an intubation procedure using an example tracheal intubation system including a laryngoscope.

FIG. 9 is a cross-sectional view of a patient after an endotracheal tube is advanced over the introducer into the field of view of the laryngoscope during an intubation procedure using an example tracheal intubation system including a laryngoscope.

FIG. 10 is a cross-sectional view of a patient after an endotracheal tube is advanced over the introducer into a final position in the trachea during an intubation procedure using an example tracheal intubation system including a laryngoscope.

FIG. 11a is a perspective view of a push-button introducer in a resting configuration.

FIG. 11b is a perspective view of a push-button introducer in a straight configuration.

FIG. 12 is an illustration of an introducer with a handle.

FIG. 13 is a perspective view of an introducer with a handle.

FIG. 14 is a side elevation view of a device that includes an introducer and a handle.

FIG. 15A is a side view of a device that includes an introducer and a handle.

FIG. 15B is a side view of a device that includes an introducer and a handle.

FIG. 16A is a top plan view of a handle in a first position.

FIG. 16B is a side elevation view of the handle of FIG. 16A in the first position.

FIG. 16C is a top plan view of the handle of FIG. 16A in a second position.

FIG. 16D is a side elevation view of the handle of FIG. 16A in the second position.

FIG. 17A is a top plan view of a handle in a first position.

FIG. 17B is a side elevation view of the handle of FIG. 17A in the first position.

FIG. 17C is a top plan view of the handle of FIG. 17A in a second position.

FIG. 17D is a side elevation view of the handle of FIG. 17A in the second position.

FIG. 18A is a top plan view of a handle in a first position.

FIG. 18B is a side elevation view of the handle of FIG. 18A in the first position.

FIG. 18C is a top plan view of the handle of FIG. 18A in a second position.

FIG. 18D is a side elevation view of the handle of FIG. 18A in the second position.

FIG. 19A is a top plan view of a handle in a first position.

FIG. 19B is a side elevation view of the handle of FIG. 19A in the first position.

FIG. 19C is a top plan view of the handle of FIG. 19A in a second position.

FIG. 19D is a side elevation view of the handle of FIG. 19A in the second position.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

The present disclosure relates generally to an introducer that is usable with a tracheal intubation system. An introducer is a slender probe that is used to guide placement of an endotracheal tube. Introducer are also sometimes referred to a stylet or catheter. This disclosure also relates to methods of performing tracheal intubation and endotracheal tube exchange procedures.

Introducers are used to help guide an endotracheal tube into place in a patient. It can be difficult to place an endotracheal tube in patients who have abnormal airways, are overweight, have undergone trauma, have arthritis, have had cervical fusions, or are combative. An introducer helps place an endotracheal tube in a patient when placing the endotracheal tube independently it otherwise not possible.

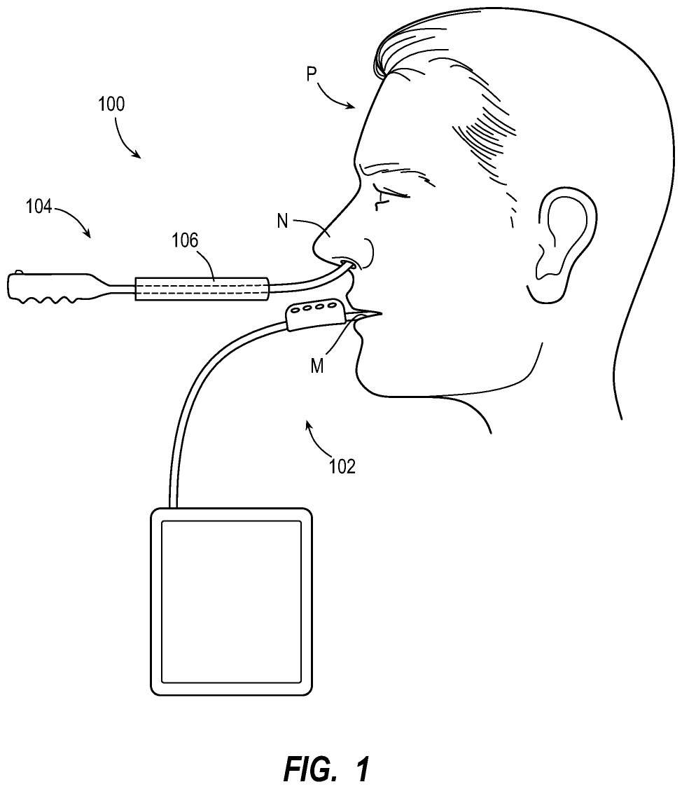

FIG. 1 is a diagram of an example tracheal intubation system 100 including a laryngoscope being used to intubate a patient P. The example intubation system 100 includes a laryngoscope 102, an introducer 104, and an endotracheal tube 106. Also illustrated are the mouth M and the nose N of the patient P. In this example, the laryngoscope 102 is inserted into the mouth M of the patient P, the introducer 104 is inserted into the nose N of the patient P, and the endotracheal tube 106 is mounted on the introducer 104. Alternatively, the introducer 104 is inserted into the mouth M of the patient P.

The patient P is a person or animal who is being intubated. Although the intubation system 100 is particularly useful to intubate a patient with a difficult airway, the intubation system 100 may also be used on a patient with a normal airway. Examples of patient P include adults, children, infants, elderly people, obese people, people with tumors affecting the head or neck, and people with unstable cervical spines. In some embodiments, the intubation system 100 may be used to intubate animals with normal or difficult airways. The intubation system 100 may be used to intubate other people or animals as well.

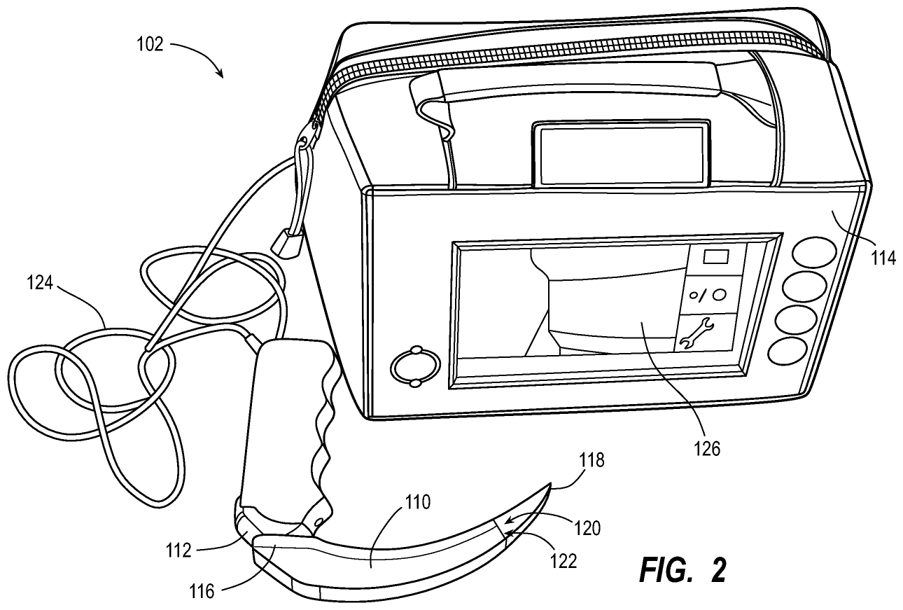

The laryngoscope 102 is a medical instrument configured to permit a medical professional to directly or indirectly view, among other things, the glottis of the patient P. In some embodiments, the laryngoscope 102 includes a blade with an integrated optical capture device and light source. In some embodiments, the blade is configured to be inserted through the mouth M of the patient P and positioned so that the glottis is in the field of view of the optical capture device. The image captured by the laryngoscope 102 is viewed from a position that is external to the patient P. In some embodiments, the image captured by the laryngoscope 102 is viewed on an external display device, such as a screen. The laryngoscope 102 is illustrated and described in more detail with reference to FIG. 2.

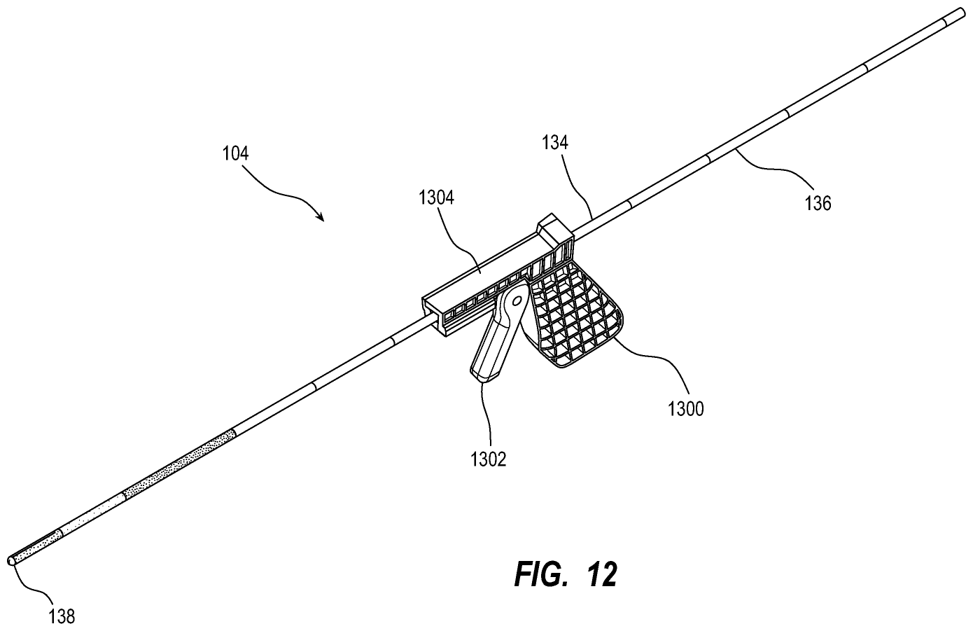

The introducer 104 is a device that is inserted into the patent P's airway. In this example, the introducer 104 is used to guide the placement of the endotracheal tube 106. The introducer 104 includes a thin, flexible tube that may be directed and advanced into the airway of the patient P. The introducer 104 may be configured to be viewed with the laryngoscope 102 during the intubation procedure. The introducer 104 is illustrated and described in more detail throughout the application, including with reference to FIGS. 12-33.

In some embodiments, the endotracheal tube 106 is a hollow tube that is configured to be placed in the airway of the patient P. When the patient P is intubated, one end of the endotracheal tube 106 is disposed inside the trachea of the patient P and the other end is connected to an external ventilator or breathing circuit. The endotracheal tube 106 is configured to occlude the airway of the patient P. Thus, gases (e.g., room air, oxygenated gases, anesthetic gases, expired breath, etc.) may flow into and out of the trachea of the patient P through the endotracheal tube 106. In some embodiments, the endotracheal tube 106 may be connected to a breathing circuit, including for example a machine-powered ventilator or a hand-operated ventilator. In other embodiments, the patient P may breathe through the endotracheal tube 106 spontaneously. The endotracheal tube 106 is illustrated and described in more detail with reference to FIG. 4.

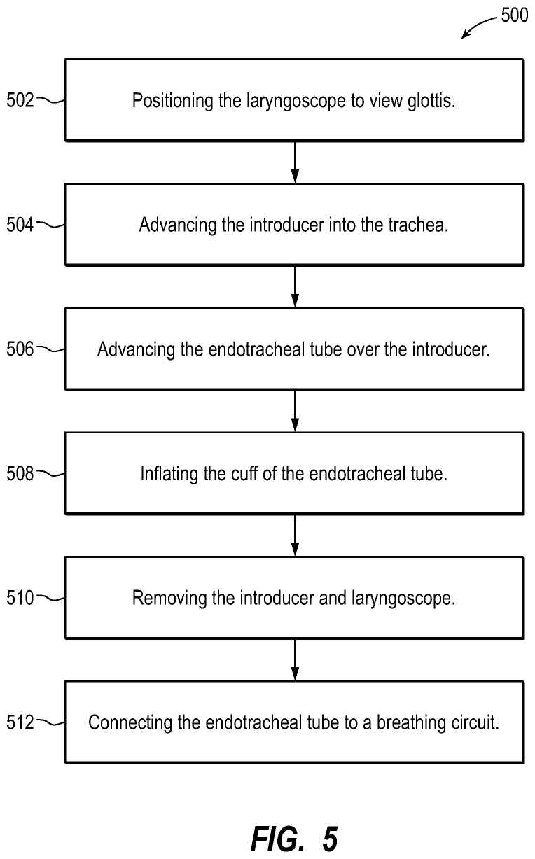

The endotracheal tube 106 is configured to be mounted on the introducer 104 by sliding over the tip and along the shaft of the introducer 104. After a medical professional has positioned the tip of the introducer 104 in the trachea of the patient P, the endotracheal tube 106 is advanced over the shaft of the introducer 104 and into the trachea of the patient P. In this manner, the introducer 104 guides the endotracheal tube 106 into the proper location in the trachea of the patient P. The process of positioning the endotracheal tube 106 is illustrated and described in more detail with reference to FIGS. 5-11.

FIG. 2 is a perspective view of an example of the laryngoscope 102. In some embodiments, the laryngoscope 102 includes a blade 110, handle 112, and display device 114.

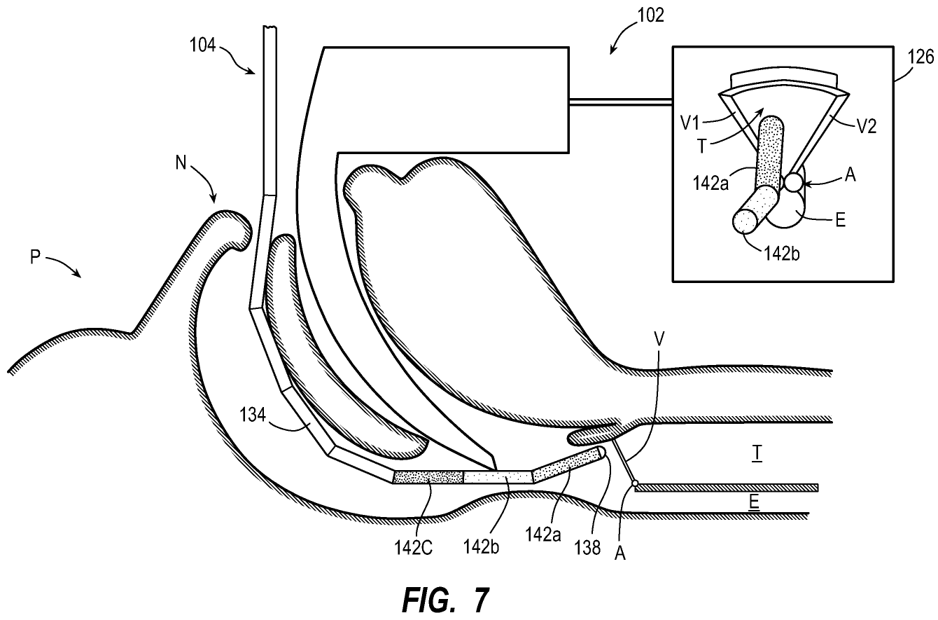

In some embodiments, the blade 110 is curved and has a first end 116 and a second end 118. The first end 116 is coupled to the handle 112. The second end 118 is configured to be inserted through the mouth of the patient and into the pharynx of the patient as illustrated and described with reference to FIG. 7. In some embodiments, the blade 110 is straight. In some embodiments, the cross section of the blade 110 is trough-like, while in other embodiments the cross section of the blade 110 is tubular. Yet other embodiments of the blade 110 are possible.

In some embodiments, the blade 110 includes an optical capture device 120 and light source 122. In some embodiments, the optical capture device 120 and the light source 122 are disposed near the second end 118 of the blade 110. Accordingly, when the blade 110 is inserted into the pharynx of the patient, the light source 122 illuminates the glottis of the patient and the optical capture device 120 captures an optical representation of the glottis of the patient, such as an image, a video, or light waves. In some embodiments, the blade 110 includes multiple optical capture devices 120 and light sources 122.

The optical capture device 120 is a device for capturing images. In some embodiments, the optical capture device 120 is a camera or image capture sensor, such as a charge-coupled device or complementary metal-oxide-semiconductor. In some embodiments, the optical capture device 120 is a digital video camera. In other embodiments, the optical capture device 120 is an optical fiber. In yet other embodiments, the optical capture device 120 is a mirror. Yet other embodiments of the optical capture device 120 are possible as well.

The light source 122 is a device that is configured to transmit or direct light towards the glottis. In some embodiments, the light source 122 is configured to generate light. In other embodiments, the light source 122 is configured to reflect light. Examples of the light source 122 include light emitting diodes, incandescent bulbs, optical fibers, and mirrors. Other embodiments include other light sources.

The handle 112 is coupled to the first end 116 of the blade 110 and is configured to be held in a hand of a user. The user may be an autonomous robot, a semi-autonomous robot, a robot remotely controlled by a medical professional, or a medical professional. Throughout the specification, any user is referred to as a medical profession, which is not intended to be limiting. The handle 112 operates to receive inputs from a medical professional and to adjust the position and orientation of the blade 110, and accordingly to aim the optical capture device 120 contained at the second end 118 thereof.

In some embodiments, the handle 112 has a cylindrical shape. In some embodiments, the cross section of the handle 112 is rectangular. In other embodiments, the cross section of the handle 112 is rectangular with rounded corners. In some embodiments, the handle 112 includes one or more molded finger grips. Other embodiments have other configurations of handle 112.

The display device 114 is configured to display, among other things, videos, images, or light waves that are captured by the optical capture device 120. In some embodiments, the display device 114 includes a screen 126. In some embodiments, the display device 114 is coupled to the handle 112 with a cable 124. In other embodiments, the display device 114 is formed integrally with the handle 112. In some embodiments, the display device 114 is a mirror. In some embodiments, a single mirror operates as both the display device 114 and the optical capture device 120. Yet other embodiments of display device 114 are possible.

In some embodiments, a cable 124 is disposed inside part or all of the handle 112, the blade 110, or both. In some embodiments, the cable 124 is configured to carry power to the optical capture device 120 and light source 122 and to carry electrical signals representing the video or images generated by the optical capture device 120 to the display device 114. In other embodiments, cable 124 is a fiber cable and operates to optically transmit light waves captured by the optical capture device 120 to the display device 114. Other embodiments do not include cable 124. For example, in some embodiments, video or images captured by the optical capture device 120 are transmitted wirelessly to the display device 114. In yet other embodiments, images captured by the optical capture device 120 are transmitted with one or more mirrors.

In some embodiments, the screen 126 is a liquid crystal display. In other embodiments, the screen 126 is a light-emitting diode display or cathode ray tube. In some embodiments, screen 126 is the surface of a mirror. Still other embodiments of the screen 126 are possible as well. The screen 126 operates to receive a signal representing an image and display that image.

Examples of the laryngoscope 102 include the GLIDESCOPE® video laryngoscope, manufactured by Verathon Inc. of Bothell, Wash., the VIVIDTRAC VT-A100® video intubation device, manufactured by Vivid Medical Inc. of Palo Alto, Calif., and the C-MAC® video laryngoscope, manufactured by Karl Storz GmbH & Co. KG of Tuttlingen, Germany. Other examples of laryngoscope 102 include other video laryngoscopes, fiber optic bronchoscopes, fiber optic stylets, mirror laryngoscopes, and prism laryngoscopes. There are many other examples of the laryngoscope 102 as well.

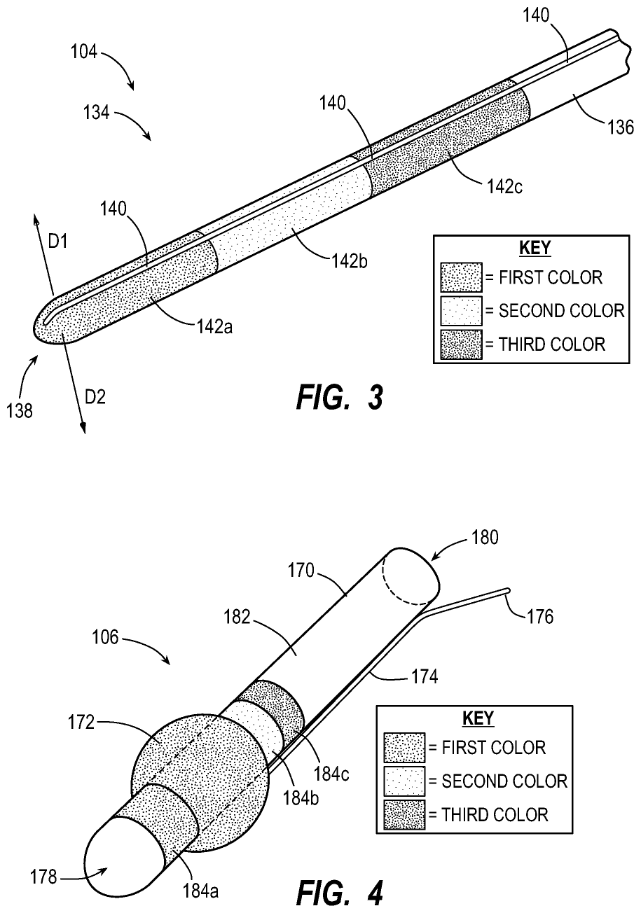

FIG. 3 is a perspective view of an example introducer 104 configured to guide an endotracheal tube into the trachea of a patient. The introducer 104 includes a handle (not shown), shaft 134, and tip 138. The shaft 134 includes an exterior surface 136 and a tip 138. The shaft 134 is configured to be inserted into the nose or mouth of a patient and directed through the glottis of the patient and into the trachea of the patient.

At an end opposite the tip 138, the shaft 134 may be coupled to the handle (not shown). In some embodiments, the shaft 134 is between two to three feet in length and has a diameter of 3/16 of an inch. In other embodiments, especially those directed towards pediatric patients, the shaft 134 has a smaller diameter. Other embodiments, with smaller or greater lengths or smaller or greater diameters are possible as well.

In some embodiments, the shaft 134 has a tubular shape and is formed from a flexible material that is configured to adapt to the shape of the airway of the patient. In some embodiments, the cross-section of the shaft 134 has an oblong shape. Other embodiments of shaft 134 with other shapes are possible.

In some embodiments, the exterior surface 136 comprises a single, continuous, uniform material. In some embodiments, the exterior surface 136 has non-stick properties. For example, in some embodiments the exterior surface 136 is formed from polytetrafluoroethylene. In other embodiments, the exterior surface 136 is configured to receive a lubricant. Other embodiments of the exterior surface 136 are possible as well. Because the exterior surface 136 is formed from a continuous material, the exterior surface 136 does not have any seams. Accordingly, the exterior surface 136 can be quickly and inexpensively cleaned. For example, the exterior surface 136 may be sterilized without the use of expensive and time-consuming sterilization equipment (e.g., an autoclave).

In some embodiments, the tip 138 is configured to minimize trauma as it moves through the nose or mouth into the upper airway and advances into the trachea of the patient. In some embodiments, the tip 138 is contained within the exterior surface 136. In some embodiments, the tip 138 has a blunt rounded shape. In some embodiments, the tip 138 does not have edges, corners, or crevices that may potentially injure the patient. Still other embodiments of the tip 138 are possible.

In some embodiments the shaft 134 and tip 138 do not contain, and are free of, a camera, light source, or other mechanism to illuminate or capture images of the patient. Accordingly, in some embodiments the design of the exterior surface 136 of the shaft 134 and tip 138 is designed to reduce trauma and simplify sterilization. The design of the exterior surface 136 of the shaft 134 and tip 138 is not constrained by the requirements of a camera, light source, or optical fibers, such as lenses, heating elements for defogging, and lumens for directing water or suctioning to clear the field of view.

The orientation mark 140 is an indicator that is on or visible through the exterior surface 136 and is configured to be visible when the introducer 104 is viewed with the laryngoscope 102. The orientation mark 140 is configured to convey qualitative information about the radial orientation of the introducer 104. In some embodiments, quantitative information may be conveyed as well. In some embodiments, the orientation mark 140 is a straight line that starts at or near the end of tip 138 and continues longitudinally along the length of shaft 134. In some embodiments, the orientation mark 140 is present throughout the entire length of the shaft 134. In other embodiments, the orientation mark 140 is only present along a portion of the shaft 134. In some embodiments, the orientation mark 140 is radially aligned with the direction D1, in which the tip 138 is configured to move. In this manner, a medical professional is able to view the orientation mark 140 on the display device of the laryngoscope 102 to determine the direction the tip 138 will move if it is pivoted. Thus, a medical professional is able to quickly direct the introducer 104 into the trachea of the patient without erroneously pivoting the tip 138, which may result in delay or trauma to the patient. In other embodiments, the orientation mark 140 is absent.

In some embodiments, the orientation mark 140 is a dashed line or a series of dots. In some embodiments, the orientation mark 140 is not radially aligned with the direction D1 but still conveys the orientation information necessary for a medical professional to direct the introducer 104. In some embodiments, multiple orientation marks are included. Yet other embodiments are possible as well.

In some embodiments, the introducer 104 includes one or more depth-assessment bands 142. In the embodiment shown in FIG. 3, the introducer 104 includes a first depth-assessment band 142a, second depth-assessment band 142b, and a third depth-assessment band 142c. The depth-assessment bands 142 are visual indicators that are on or visible through the exterior surface 136 and are configured to be visible when the introducer 104 is viewed with the laryngoscope 102. The depth-assessment bands 142 are configured to convey qualitative information about the placement of the introducer 104 relative to the anatomical landmarks of the patient, such as the vocal cords, that are also visible through the laryngoscope 102. In some embodiments, quantitative information may be conveyed as well. The depth-assessment bands 142 are also configured to convey both qualitative and/or quantitative information about the longitudinal distance to the end of the tip 138.

Adjacent depth-assessment bands 142 are visually distinct from each other so that a medical professional who views a part of one of the depth-assessment bands 142 from the laryngoscope is able to identify specifically which of the depth-assessment bands 142 is in the field of view. Because the depth-assessment bands 142 are continuous regions, it is not necessary for a medical professional to advance or retract the introducer 104 to bring one of the depth-assessment bands 142 into the field of view of the laryngoscope 102, which would create a risk of trauma to the patient or inadvertent removal of the introducer 104 from the trachea of the patient. For example, patients with endotracheal tubes may require a chest x-ray to determine the tip depth of the endotracheal tube, when depth assessment bands are not present. This process may be time consuming, costly and may require patient movement in order to obtain the chest x-ray. Patient movement during performance of a chest x-ray is a leading cause of accidental extubation. It may be therefore advantageous to design equipment to confirm the depth of endotracheal tubes, introducers or other devices in the patent's trachea that does not require chest x-rays. Nor does a medical professional need to remember or count the depth-assessment bands 142 as they pass through the field of view. In this manner, the depth-assessment bands 142 minimize trauma to the patient and allow a medical professional to focus on using the introducer 104 rather than counting depth-assessment bands 142. Further, using the depth-assessment bands 142 in this manner may reduce the time necessary to complete a tracheal intubation procedure.

In some embodiments, the depth-assessment bands 142 are continuous regions of color that extend along a portion of the length of the shaft 134. For example, the first depth-assessment band 142a is a first color, the second depth-assessment band 142b is a second color, and the third depth-assessment band 142c is a third color. In other embodiments, the depth-assessment bands 142 are continuous regions of visually distinct patterns rather than colors. In some embodiments, the depth-assessment bands 142 include both visually distinct patterns and colors. Yet other embodiments are possible as well.

In some embodiments, the lengths of the depth-assessment bands 142 are selected based on the clinical precision required for the intubation procedure in which the introducer 104 is intended and the distance into the trachea of the patient, a medical professional wishes to insert the tip 138. For example, a medical professional may wish to insert the tip 138 two to four centimeters into the trachea of an adult patient. In some embodiments for adult patients, the length of each of the depth-assessment bands 142 is two centimeters. In this manner, the medical professional will know that the tip 138 is properly inserted into the trachea of the patient when any part of the second depth-assessment band 142b is aligned with the entrance of the trachea of an adult patient (i.e., the patient's vocal cords). In another example, the medical professional may not know or be able to recall the safe distance of insertion into the trachea for an adult patient in numeric or quantitative form. In some embodiments, this safe depth is embedded in the design of the visually distinct colors or patterns of the depth-assessment band. This allows the medical professional to achieve safe depth of placement using a qualitative methodology by aligning a one or more distinctly visible depth assessment bands up with an anatomic marker. (i.e., the patient's vocal cords).

Similarly, in some embodiments for pediatric patients, the lengths of the depth-assessment bands 142 are adapted to the shorter tracheas of those pediatric patients. For example, a medical professional may wish to insert the tip 138 one to two centimeters into the trachea of the pediatric patient. In some embodiments for pediatric patients, the length of each depth-assessment band 142 is one centimeter. In this manner, the medical professional will know that the tip 138 is properly inserted into the trachea of the patient when any part of the second depth-assessment band 142b is aligned with the entrance of the trachea of a pediatric patient (i.e., the patient's vocal cords). In another example, the medical professional may not know or be able to recall the safe distance of insertion into the trachea for a pediatric patient in numeric or quantitative form. In some embodiments, this safe depth is embedded in the design of the visually distinct colors or patterns of the depth-assessment band. This allows the medical professional to achieve safe depth of placement using a qualitative methodology by aligning a one or more distinctly visible depth assessment bands up with an anatomic marker. (i.e., the patient's vocal cords).

In some embodiments, the colors of the depth-assessment bands 142 convey information about whether the tip 138 is properly positioned. In some example embodiments, the first depth-assessment band 142a is yellow, the second depth-assessment band 142b is green, and the third depth-assessment band 142c is red. The yellow color of the first depth-assessment band 142a may convey to a medical professional to use caution in advancing the tip 138 because it is not yet properly positioned. The green color of the second depth-assessment band 142b may convey success to a medical professional because the tip 138 appears to be properly positioned. The red color of the third depth-assessment band 142c may convey warning to a medical professional because the tip 138 may be positioned too deeply in the trachea of the patient, potentially causing trauma.

Although the embodiment shown in FIG. 3 includes three depth-assessment bands 142, other embodiments that include fewer or more depth-assessment bands 142 are possible as well. In some embodiments, the depth-assessment bands 142 are uniform in length. In other embodiments, one or more of the depth-assessment bands 142 has a different length than the other depth-assessment bands 142. For example, in applications requiring great precision, one of the depth-assessment bands 142 is shorter in length than the other depth-assessment bands 142. Accordingly, when that one of the depth-assessment bands 142 is aligned with the entrance to the trachea of a patient (i.e., the vocal cords), a medical professional is able to determine the depth of the tip 138 with greater precision.

Although the embodiment of the depth-assessment bands 142 shown in FIG. 3 relates to an introducer 104, the depth-assessment bands 142 can also be used with other introducers, stylets, exchange catheters, and/or endotracheal tubes. For example, in some embodiments, the depth-assessment bands 142 are used with an introducer that is not malleable. In these embodiments, the introducer is similar to the introducer 104 described herein, except that the tip articulates and components that control the tip are included. In these embodiments, the introducer still includes the depth-assessment bands 142, which can be viewed with the laryngoscope 102 to determine the position of the non-articulating tip of the introducer relative to various anatomical landmarks. While it may be advantageous to move the tip in various directions about a single point on the shaft of the device, this single point of tip/shaft articulation does not always allow easy tube delivery as multiple angles varying in degree and orientation may be needed at various points along the shaft and tip to allow easy navigation into an airway with a tortuous pathway through the upper airway, to the entrance of the trachea. It may be also advantageous to be able to dynamically change the shape of the shaft and tip at multiple points along the shaft and tip allowing navigation of the airway into the trachea, allowing it to wind its way through a tortuous pathway.

Although the embodiments described herein relate to placement of an endotracheal tube, the depth-assessment bands that convey quantitative and/or qualitative depth information are not limited to use in airway devices. In some embodiments, the depth-assessment bands 142 are included on other medical devices to guide the proper placement of those medical devices as well. For example, in some embodiments, the depth-assessment bands 142 are included in central venous catheters, endoscopic devices, devices placed in the gastrointestinal tract, devices placed inside the cardiovascular system, devices placed inside the urinary system, devices placed inside of the ears, devices placed inside of the eyes, devices placed in the central nervous system, devices placed inside of the abdomen, devices placed inside the chest, or devices placed inside the musculoskeletal system. In these embodiments, the depth-assessment bands 142 are configured to be compared to various tissue structures. In these embodiments, the depth-assessment bands 142 are configured to convey quantitative and/or qualitative information about the placement of the device relative to various anatomical landmarks compared to other organ systems inside the body or even outside of the body. Additionally, in some embodiments, the depth-assessment bands 142 are included on non-medical devices in which depth control is desired. For example, the depth-assessment bands 142 can be included in industrial devices, such as devices for the inspection of machinery or physical structures, and devices for the proper placement of fasteners or other industrial or physical parts.

FIG. 4 is a perspective view of an example endotracheal tube 106. The endotracheal tube 106 includes a pipe 170, a cuff 172, and an inflation lumen 174. In some embodiments, the endotracheal tube 106 does not include the cuff 172 or the inflation lumen 174.

In some embodiments, the pipe 170 is hollow and includes a first end 178, a second end 180, and an exterior surface 182. In some embodiments, the pipe 170 is formed from a flexible material and operates to adapt to the anatomy of the patient. For example, in some embodiments, the pipe 170 is formed from polyvinyl chloride. In other embodiments, the pipe 170 is formed from silicone rubber or latex rubber. In some embodiments, the pipe 170 is formed from a rigid or semi-rigid material, such as stainless steel.

The pipe 170 operates as a passage for gases to enter and exit the trachea of the patient. The pipe 170 also operates to protect the lungs of the patient from stomach contents. Further, in some embodiments, the pipe 170 operates as a passage to suction the trachea and lungs of the patient. The first end 178 is configured to be advanced into the trachea of the patient. The second end 180 is configured to be connected to a ventilator or breathing circuit.

In some embodiments, the cuff 172 is disposed on the exterior surface 182 of the pipe 170 near the first end 178. The cuff 172 is configured to form a seal between the exterior surface 182 of the pipe 170 and the trachea of the patient. In this manner, the cuff 172 prevents gases and liquids from entering or exiting the trachea of the patient without passing through the pipe 170. In addition, the cuff 172 secures the position of the endotracheal tube 106 in the trachea of the patient. In some embodiments, the cuff 172 is an inflatable chamber. For example, in some embodiments, the cuff 172 is a balloon. Yet other embodiments of the cuff 172 are possible as well.

The inflation lumen 174 includes an inflation port 176. The inflation lumen 174 is connected to the cuff 172 and operates as a channel for the entry of fluid into the cuff 172. The inflation port 176 is configured to receive a fluid. In some embodiments, the inflation port 176 is configured to receive a syringe that operates to expel fluid through the inflation lumen 174 and into the cuff 172. In this manner, the cuff 172 can be inflated to seal the trachea of the patient.

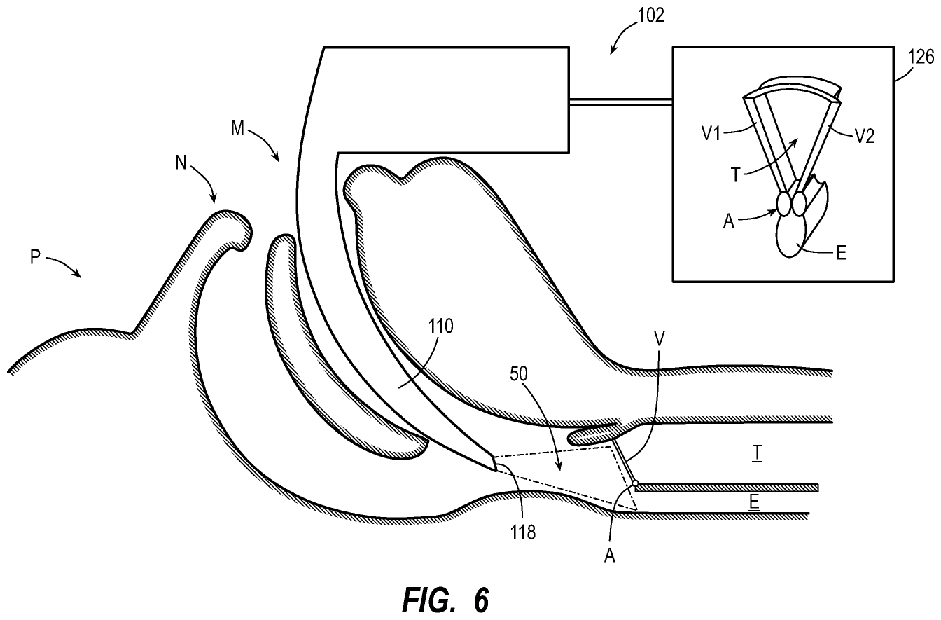

In some embodiments, the endotracheal tube 106 is formed from a transparent or translucent material that allows the introducer 104 to be seen there through. In some embodiments, the endotracheal tube 106 includes one or more depth-assessment bands 184a-c (collectively depth-assessment bands 184). In the embodiment shown in FIG. 6, the example endotracheal tube 106 includes a first depth-assessment band 184a, second depth-assessment band 184b, and a third depth-assessment band 184c. The depth-assessment bands 184 are indicators that are on or visible through the exterior surface 182 and are configured to be visible when the introducer 104 is viewed with the laryngoscope 102. The depth-assessment bands 184 are configured to convey information about the placement of the endotracheal tube 106 relative to the anatomical landmarks of the patient, such as the vocal cords, that are also visible through the laryngoscope 102. The depth-assessment bands 184 are also configured to convey information about the longitudinal distance to the end of the first end 178.

Adjacent depth-assessment bands 184 are visually distinct from each other so that a medical professional who views a part of one of the depth-assessment bands 184 from the laryngoscope 102 is able to identify which specific one of the depth-assessment bands 184 is in the field of view. Because the depth-assessment bands 184 are continuous regions, it is not necessary for a medical professional to advance or retract the endotracheal tube 106 to bring the depth-assessment bands 184 into the field of view of the laryngoscope 102, which would create a risk of trauma to the patient or inadvertent removal of the endotracheal tube 106 from the trachea of the patient. Nor does a medical professional need to remember or count the depth-assessment bands 184 as they pass through the field of view. In this manner, the depth-assessment bands 184, minimize trauma to the patient and allow a medical professional to focus on advancing the endotracheal tube 106 rather than counting depth-assessment bands 184. Further, using the depth-assessment bands 184, in this manner may reduce the time necessary to complete a tracheal intubation procedure.

In some embodiments, the depth-assessment bands 184 are continuous regions of color that extend along a portion of the length of the pipe 170. For example, the first depth-assessment band 184a is a first color, the second depth-assessment band 184b is a second color, and the third depth-assessment band 184c is a third color. In other embodiments, the depth-assessment bands 184 are continuous regions of visually distinct patterns rather than colors. In some embodiments, the depth-assessment bands 184 include both visually distinct patterns and colors. In addition, in some embodiments, one or more of the depth-assessment bands 184 may include part or all of cuff 172. Yet other embodiments of the depth-assessment bands 184 are possible as well.

In some embodiments, the lengths of the depth-assessment bands 184 are selected based on the clinical precision required for the intubation procedure in which the endotracheal tube 106 is intended and the distance into the trachea of the patient, a medical professional wishes to insert the first end 178. For example, a medical professional may wish to insert the first end 178 two to four centimeters into the trachea of an adult patient. In some embodiments for adult patients, the length of each of the depth-assessment bands 184 is two centimeters. In this manner, the medical professional will know that the first end 178 is properly inserted into the trachea of the patient when any part of the second depth-assessment band 184b is aligned with the entrance of the trachea of an adult patient (i.e., the patient's vocal cords). In another example, the medical professional may not know or be able to recall the safe distance of insertion into the trachea for an adult patient in numeric or quantitative form. In some embodiments, this safe depth is embedded in the design of the visually distinct colors or patterns of the depth-assessment band. This allows the medical professional to achieve safe depth of placement using a qualitative methodology by aligning a one or more distinctly visible depth assessment bands up with an anatomic marker. (i.e., the patients vocal cords)

Similarly, in some embodiments for pediatric patients, the lengths of the depth-assessment bands 184 are adapted to the shorter tracheas of those pediatric patients. For example, a medical professional may wish to insert the first end 178 one to two centimeters into the trachea of the pediatric patient. In some embodiments for pediatric patients, the length of each of the depth-assessment bands 184 is one centimeter. In this manner, the medical professional will know that the first end 178 is properly inserted into the trachea of the patient when any part of the second depth-assessment band 184b is aligned with the entrance of the trachea of a pediatric patient (i.e., the patient's vocal cords). In another example, the medical professional may not know or be able to recall the safe distance of insertion into the trachea for a pediatric patient in numeric or quantitative form. In some embodiments, this safe depth is embedded in the design of the visually distinct colors or patterns of the depth-assessment band. This allows the medical professional to achieve safe depth of placement using a qualitative methodology by aligning a one or more distinctly visible depth assessment bands up with an anatomic marker. (i.e., the patient's vocal cords).

In some embodiments, the colors of the depth-assessment bands 184 convey information about whether the first end 178 is properly positioned. In some example embodiments, the first depth-assessment band 184a is yellow, the second depth-assessment band 184b is green, and the third depth-assessment band 184c is red. The yellow color of the first depth-assessment band 184a may convey to a medical professional to use caution in advancing the first end 178 because it is not yet properly positioned. The green color of the second depth-assessment band 184b may convey success to a medical professional because the first end 178 appears to be properly positioned. The red color of the third depth-assessment band 184c may convey warning to a medical professional because the first end 178 may be positioned too deeply in the trachea of the patient, potentially causing trauma.

Although the embodiment shown in FIG. 6 includes three depth-assessment bands 184, other embodiments that include fewer or more depth-assessment bands 184 are possible as well. In some embodiments, the depth-assessment bands 184 are uniform in length. In other embodiments, one or more of the depth-assessment bands 184 has a different length than the other depth-assessment bands 184. For example, in applications requiring great precision, one of the depth-assessment bands 184 is shorter in length than the other depth-assessment bands 184. Accordingly, when that one of the depth-assessment bands 184 is aligned with the entrance to the trachea of a patient (i.e., the vocal cords), a medical professional is able to determine the depth of the first end 178 with greater precision.

FIG. 5 is a flowchart of an example method 500 of generally positioning an endotracheal tube in a patient using an example tracheal intubation system including a laryngoscope and an introducer.

Initially, at step 502, the laryngoscope is positioned to view the glottis of the patient. In some embodiments, the laryngoscope is inserted through the mouth of the patient. In other embodiments, the laryngoscope is inserted through the nose of the patient. A medical professional, usually a physician or a person assisting a physician, grips the handle of the laryngoscope and maneuvers the handle to position the blade so that the optical capture device of the laryngoscope has a clear view of the glottis of the patient. In some embodiments, the medical professional verifies that the laryngoscope is properly positioned by checking the screen of the display device of the laryngoscope.

At operation 504, the introducer is advanced into the pharynx of the patient until it is visible with the laryngoscope. The introducer is advanced until it is positioned in the trachea. In an example embodiment, the introducer is advanced individually into the patient. In an alternative embodiment, the endotracheal tube is mounted on the introducer before being advanced into the patient. The endotracheal tube is mounted by placing the second end of the endotracheal tube over the tip of the introducer and sliding the tube up the shaft of the introducer. This operation may be performed by the physician, someone assisting the physician, or someone preparing the equipment in advance. Alternatively, the endotracheal tube may be mounted after the introducer is placed in the trachea.

The tip of the introducer is positioned in the pharynx of the patient and is advanced until the tip is visible on the screen of the laryngoscope. In some embodiments, the tip of the introducer is inserted through the nose of the patient. In other embodiments, depending on the anatomy of the patient, the tip of the introducer is inserted through the mouth of the patient.

The tip of the introducer may be angled towards the entrance to the trachea of the patient. That is, the tip is angled so that when the introducer is advanced, the tip will pass between the vocal cords of the patient and into the trachea of the patient. In some embodiments, a medical professional, usually a physician or person assisting a physician, angles the tip of the introducer before being advanced into the patient. The medical professional angles the tip of the introducer while viewing the tip on the screen of the laryngoscope. An example embodiment of the introducer with the tip angled towards the entrance of the trachea of the patient is shown in FIG. 7.

In some embodiments, the introducer includes one or more depth-assessment bands. The medical professional views the shaft of the introducer on the screen of the laryngoscope to determine which depth-assessment band is adjacent to the vocal cords of the patient. Depending on which depth-assessment band is adjacent to the vocal cords, the medical professional may continue to advance the introducer or stop advancing the introducer.

For example, in an embodiment in which the introducer includes three depth-assessment bands and the second depth-assessment band represents the target insertion depth, a medical professional will continue to advance the introducer until the second depth-assessment band is adjacent to the vocal cords of the patient. Accordingly, if the screen of the laryngoscope shows that the first depth-assessment band is adjacent to the vocal cords, the medical professional may continue to advance the introducer. Similarly, if the screen of the laryngoscope shows that the second depth-assessment band is adjacent to the vocal cords of the patient, the medical professional may qualitatively determine that the tip of the introducer is properly positioned and, accordingly, will stop advancing the introducer. Finally, if the screen of the laryngoscope shows that the third depth-assessment band is adjacent to the vocal cords of the patient, the medical professional may determine that the tip of the introducer has been advanced too far and will stop advancing the introducer or, in some cases, will retract the introducer. An example embodiment of the introducer with three depth-assessment bands being advanced into the trachea of the patient is shown in FIGS. 7-10.

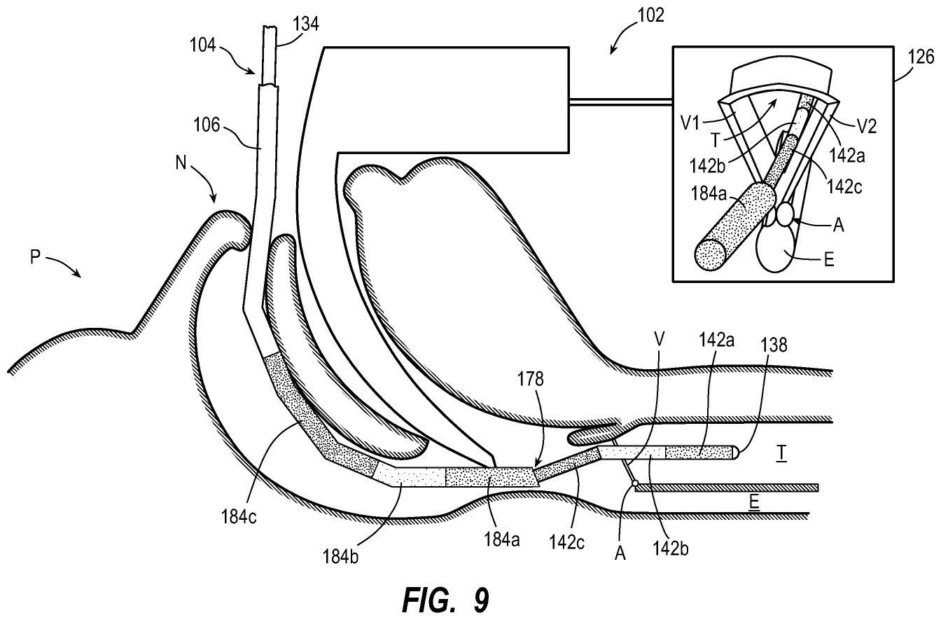

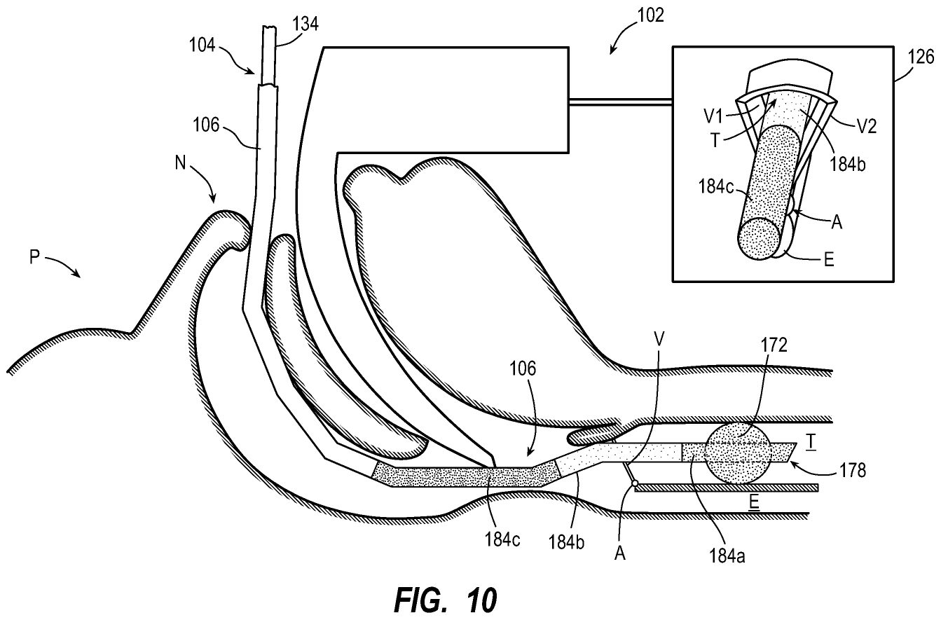

At operation 506, the endotracheal tube is advanced over the shaft of the introducer. In some embodiments, a medical professional, usually a physician or person assisting a physician grabs the endotracheal tube and slides it along the introducer until the first end of the endotracheal tube enters the trachea of the patient. An example embodiment of the endotracheal tube being advanced over the shaft of the introducer is shown in FIGS. 9 and 10.

At operation 508, the cuff of the endotracheal tube is inflated. In some embodiments, a medical professional, usually a physician or person assisting a physician inserts a fluid into the inflation port of the endotracheal tube. This causes the inflation cuff to expand and secures the endotracheal tube in the trachea of the patient. In addition, the inflation cuff seals the trachea of the patient so that gases will not flow around the endotracheal tube. Further, the inflation cuff seals the trachea of the patient so that liquids, such as the contents of the stomach of the patient, will not enter the trachea and the lungs of the patient. An example embodiment of an endotracheal tube with an inflated cuff is shown in FIG. 10. In embodiments where the endotracheal tube does not include a cuff, this operation 508 is not performed.

At operation 510, the introducer and laryngoscope are removed. The shaft of the introducer is pulled out of the endotracheal tube, leaving the endotracheal tube in place. In addition, the laryngoscope is also removed from the patient. The laryngoscope is removed by grabbing the handle and pulling the blade out of the pharynx of the patient.

At operation 512, the endotracheal tube is connected to a ventilator or breathing circuit to provide ventilation for the patient. In some embodiments, the endotracheal tube is connected to the ventilator or breathing circuit before the laryngoscope is removed.

FIG. 6 is a cross-sectional view of a patient P during an intubation procedure using an example tracheal intubation system including a laryngoscope.

The mouth M and nose N of the patient P are shown. The blade 110 of the laryngoscope 102 is disposed in the pharynx of the patient P. The blade 110 is oriented so that the field of view 50 of the optical capture device on blade 110 includes the vocal cords V and trachea T of the patient P. Screen 126 shows the contents of the field of view 50 of the optical capture device in the laryngoscope 102.

The screen 126 displays an image of the trachea T. The entrance to the trachea T is defined by the vocal cords V1 and V2 (collectively vocal cords V). The vocal cords V meet at the arytenoids A. The esophagus E is below the trachea T and parallel to the trachea T. It is important that the blade 110 of the laryngoscope 102 is oriented so that screen 126 shows a clear image of the entrance of the trachea T because the articulating stylet will be directed into the trachea T.

FIG. 7 is a cross-sectional view of a patient P during an intubation procedure using an example tracheal intubation system including a laryngoscope. The tip 138 of the introducer 104 is angled up. The screen 126 shows that the tip 138 is now directed towards the entrance of the trachea T.

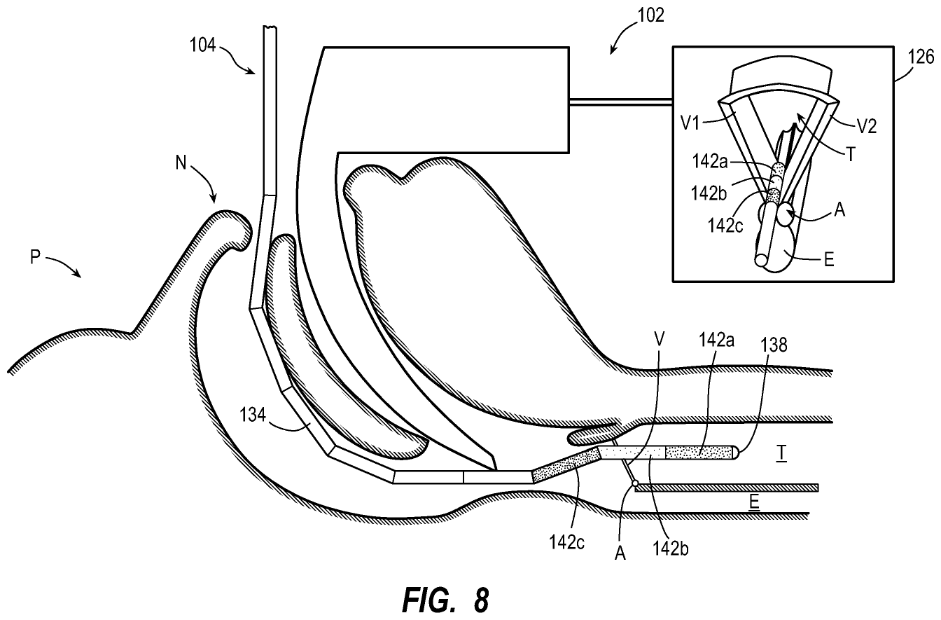

FIG. 8 is a cross-sectional view of a patient P during an intubation procedure using an example tracheal intubation system including a laryngoscope. The tip 138 of the introducer 104 is advanced further into the trachea T of the patient P as compared to FIG. 7. The screen 126 shows that the second depth-assessment band 142b is now adjacent to the vocal cords V. Accordingly, a medical professional may determine that the tip 138 is properly positioned and does not need to be advanced further into the trachea T.

FIG. 9 is a cross-sectional view of a patient P during an intubation procedure using an example tracheal intubation system including a laryngoscope. The tip 138 of the introducer 104 is properly positioned in the trachea T of the patient P. The endotracheal tube 106 has been advanced over the shaft 134 of the introducer 104. The endotracheal tube 106 is guided by the introducer 104 through the nose N of the patient P and into the pharynx of the patient P. The first end 178 and the first depth-assessment band 184a of endotracheal tube 106 are visible on the screen 126.

The tip 138 of the introducer 104 is properly positioned in the trachea T of the patient P when the second depth-assessment band 184b is adjacent to the vocal cords V. The endotracheal tube 106 is guided into the trachea T of the patient P by the introducer 104. The screen 126 displays that the first end 178 of endotracheal tube 106 has not yet reached the vocal cords V. Both the first depth-assessment band 184a and the second depth-assessment band 184b are visible on screen 125. However, neither the first depth-assessment band 184a nor the second depth-assessment band 184b are adjacent to the vocal cords V yet. Accordingly, the medical professional may determine that the first end 178 of the endotracheal tube 106 needs to be advanced further to enter the trachea T of the patient P.

FIG. 10 is cross-sectional view of a patient P during an intubation procedure using an example tracheal intubation system including a laryngoscope. The endotracheal tube 106 has been advanced further along shaft 134 of the introducer 104 as compared to FIG. 9. The screen 126 displays that the endotracheal tube 106 has entered the trachea T. Additionally, screen 126 displays that the second depth-assessment band 184b is adjacent to the vocal cords V. Accordingly, a caretaker may determine that the endotracheal tube 106 has been guided into the trachea T of the patient P and has been properly positioned therein. If instead the first depth-assessment band 184a were adjacent to the vocal cords V, a medical professional may determine that the endotracheal tube 106 needs to be advanced further into the trachea T of the patient P. Conversely, if instead the third depth-assessment band 184c were adjacent to the vocal cords V, the medical professional might determine that the endotracheal tube 106 was advanced too far into the trachea T of the patient P. Once the endotracheal tube 106 is properly positioned, the cuff 172 is inflated to seal the trachea T and secure the endotracheal tube 106 in position.

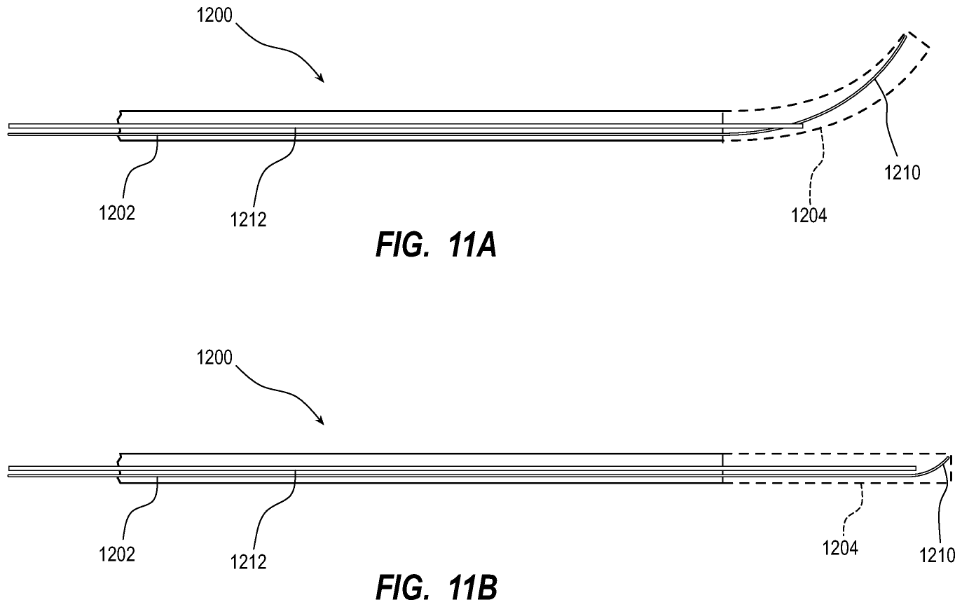

FIG. 11A is a perspective view of a push-button introducer 1200 in a resting configuration. In this example, the push-button introducer 1200 includes a shaft 1202 and a tip portion 1204. The shaft 1202 includes a lumen. The lumen may be formed as a generally round recess in an extruded plastic structure. The lumen is configured to constrain the movement of the stiffening wire 1210 and push rod 1212. Stiffening wire 1210 and push rod 1212 may extend through the same lumen, or stiffening wire 1210 may extend through a first lumen and push rod 1212 may extend through a second lumen. In a resting configuration, tip portion 1204 is curved away from a midline. As shown in FIG. 11B, when push rod 1212 is pushed through shaft 1202, tip portion 1204 straightens, or otherwise moves toward a midline.

As shown in the example of FIGS. 11A. 11B, the stiffening wire 1210 is connected to a corner of the tip portion 1204. In some embodiments, the shaft 1202 and the tip portion 1204 are formed integrally. Alternatively, the shaft 1202 and the tip portion 1204 are formed separately and joined together (e.g., with a weld such as a butt weld, an adhesive, or a coupling device).

In another embodiment, push-button introducer 1200 does not include stiffening wire 1210. In this embodiment, the shaft 1202 is more rigid than tip portion 1204. In other words, the tip portion 1204 is more flexible than the shaft 1202. For example, the shaft 1202 may be formed from a more rigid material such as a plastic having a higher durometer and the tip portion 1204 is formed from a more flexible material such as a plastic that has a lower durometer.

FIG. 12 illustrates an embodiment of an introducer 104 with a handle 1300. Introducer 104 may be an introducer as described in co-pending U.S. Patent Application No. 62/616,426, the disclosure of which is hereby incorporated by reference in its entirety. The introducer 104 may include an articulating tip. For example, the introducer 104 includes a shaft 134 having an exterior surface 136, and a tip 138. The shaft 134 is configured to be inserted into the nose or mouth of a patient and directed through the glottis of the patient and into the trachea of the patient. In some embodiments, the shaft 134 is between two to three feet in length and has a diameter of 3/16″ of an inch. In other embodiments, especially those directed towards pediatric patients, the shaft 134 has a smaller diameter. Other embodiments, with smaller or greater lengths or smaller or greater diameters are possible as well.

When passing an endotracheal tube through the glottis, the beveled tip of the endotracheal tube may catch on the glottic structures. This may be problematic for many reasons. Catching may interfere with the smooth advancement of the endotracheal tube into the trachea. Resolving the catching problem can distract the operator's attention, add mental task load to the operator, delay proper placement of the endotracheal tube into the trachea causing a delay in delivery of oxygen to the patient, and can even cause a failed intubation. Catching of the tip on the glottis may irritate or injure the glottic structures. This catching problem is especially likely when an endotracheal tube is placed into the trachea using a technique in which an introducer is first placed into the trachea and an endotracheal tube is slid over the introducer into the trachea as a gap may form between the introducer and the beveled tip of the endotracheal tube, which may increase the likelihood of catching a glottic structure in that gap. In some cases, a catheter may be used in place of an introducer.

It may be advantageous to the patient and operator to decrease the likelihood and minimize the severity of the catch problem when intubation of the trachea with any medical device. It may be advantageous to design equipment that allows an operator to view the glottic opening and its relationship to the devices being placed into the trachea throughout the procedure.

Closing the gap between the external surface of the endotracheal tube and the tip of the endotracheal tube or other devices sliding over the introducer as it passes through the glottis can decrease the chances that the glottic structures might catch on the tip of the endotracheal tube tip as it passes through the glottis.

It may be advantageous that the endotracheal tube slides as smoothly and easily as possible over the introducer guiding the endotracheal tube into the trachea during placement into the trachea. It may be advantageous to minimize or reduce surface friction between the introducer and the endotracheal tube as the endotracheal tube is passed over the introducer and into the trachea.

It may be advantageous to close the gap between the inside of the endotracheal tube tip and the outside of the introducer in the area of the glottic opening to reduce the chances of glottic catch problem while at the same time allow a gap between the inside of the endotracheal tube and the outside of the introducer along the introducer that is not interacting with the glottic opening to allow the endotracheal tube to slide as smoothly and easily over the introducer and into the trachea.

In some cases, closing the gap between the endotracheal tube tip and the introducer has been solved by either uniformly increasing the cross-sectional diameter of the entire introducer so that it fills the lumen of the endotracheal tube or bending the endotracheal tube tip inward so that it rides along the surface of the introducer acting to close the gap as it rides down the introducer.

Both of these solutions present other clinical problems that would be advantageous to solve. Uniformly increasing the cross-sectional diameter of the introducer to fill endotracheal tube lumen increases surface contact between the introducer and endotracheal tube making it more difficult to slide over the introducer into the trachea. Specialized endotracheal tubes with inwardly bending tips must be immediately available for use, which may not be the case in all care settings.

An introducer with variable cross-sectional diameters may solve this problem. Where the introducer is not interacting with the glottis, the cross-sectional diameter of the introducer may be smaller to minimize surface friction between the introducer and the endotracheal tube as the endotracheal tube is passed over the introducer and into the trachea. Where the introducer is interacting with the glottis, the cross-sectional diameter of the introducer may be larger to close the gap between the inside of the endotracheal tube tip and the outside of the introducer in the area of the glottic opening to reduce the chances of glottic catch problem. An introducer with variable cross-sectional diameters can minimize the glottic catch problem while at the same time maximize the overall surface contact between the introducer and endotracheal tube.

It may be advantageous for the tip of an airway introducer to be small, as it is easier to place a small tip into a glottic opening than a large tip into the same size glottic opening. It may be advantageous to have an airway introducer taper from a small diameter at the tip to a larger diameter so that it is easy to place the introducer into the glottic opening. Once the tip is passed into the trachea to a certain proper depth, it may be advantageous to have the portion of the shaft in the glottic opening be a larger diameter, or taper to be a larger diameter. That larger diameter could be the proper diameter to fill the inside of the endotracheal tube, thereby closing the gap between the endotracheal tube tip and the shaft of the introducer lying at the glottis in order to decrease the risk of the tip of the tube catching on the glottis.

Shaft 134 may be coupled to a handle 1300. Handle 1300 may be located at a midpoint of shaft 134, or at a location that is far enough from tip 138 so introducer 104 may be placed in patent at an adequate depth. Handle 1300 may be permanently affixed to shaft 134, or alternatively, shaft 134 may be removable from introducer 104. Handle 1300 includes connection mechanism 1304 that allows handle 1300 to be connected to shaft 134. The connection mechanism 1304 allows the handle 1300 to be easily and quickly removed from the introducer 104. In use, an endotracheal tube is able to pass over the introducer 104 after the handle 1300 has been removed from the introducer 104. Handle 1300 is configured to articulate the tip 138 of the introducer 104 once positioned in the patient.

In an embodiment, handle 1300 includes a trigger 1302, which when actuated, causes the tip 134 to articulate. Other articulating mechanisms may include a scissors-type handles, a ratchet mechanism, tension spring, or other similar articulating mechanisms.

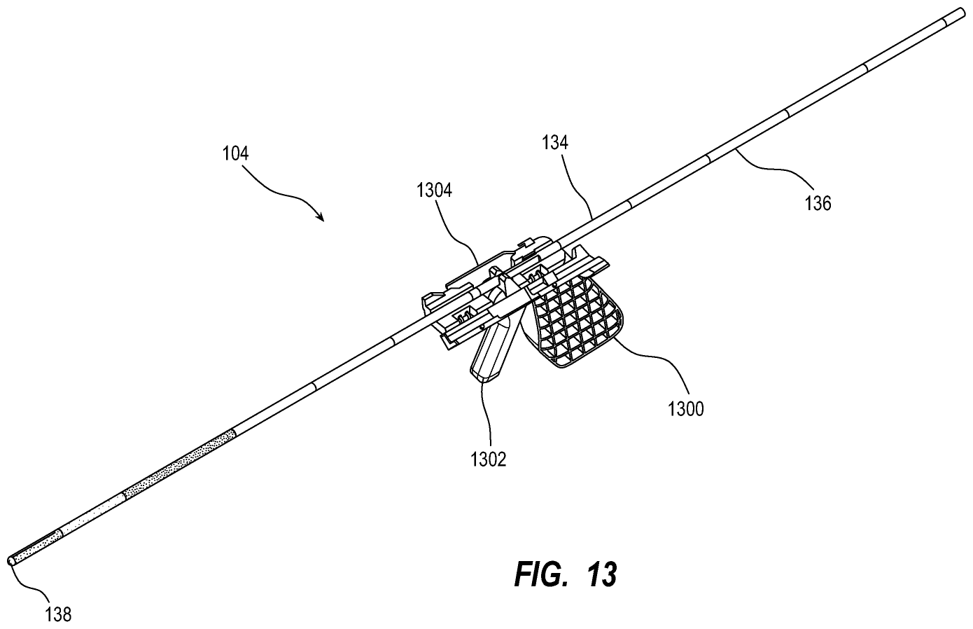

FIG. 13 illustrates an example embodiment of the inside of the connection mechanism 1304 of the handle 1300. Connection mechanism 1304 is configured to articulated tip 138. A connection mechanism 1304 may be a friction fit, snap-fit, or other similar types of locking mechanisms.

An example method of using an introducer 104 with a handle 1300 includes the following. First, an introducer 104 is loaded onto the handle 1300. Next, a laryngoscope is placed into the mouth of the patient and advanced until a view of the glottis is obtained. The introducer is placed into the patient's mouth and is guided through the vocal cords. The introducer is advanced until the green zone of the color depth zones lies adjacent to the glottis. Once the introducer is at the appropriate location, it is held in place and the handle is used to articulate the tip of the introducer. The tip is articulated in an anterior direction by the handle.

Once the introducer is at the appropriate location the handle is removed from the introducer, and the introducer remains properly placed in the trachea. Next, an endotracheal tube is placed on the proximal end of the introducer and is advanced over the introducer into the trachea. Once the distal tip of the new endotracheal tube reaches the glottis, the medical professional can observe the tip of the endotracheal tube to pass smoothly through the glottis over the introducer. Throughout advancing the endotracheal tube, the medical professional continually keeps the green zone of the introducer at the glottis.

The green zone of the introducer should remain adjacent to the glottis while the endotracheal tube is advanced. If the yellow zone of the introducer is adjacent to the glottis, the medical professional advances the introducer further into the patient's trachea until the green zone is adjacent the glottis again. If the red zone of the introducer is adjacent to the glottis, the medical professional retracts the introducer from the patient's trachea until the green zone is adjacent the glottis again.

Once the distal tip of the endotracheal tube is properly placed in the trachea, the introducer is removed from the endotracheal tube, while the endotracheal tube remains properly positioned in the trachea. The endotracheal tube can then be inflated and connected to the ventilator. Finally, the laryngoscope can be removed from the patient.

In an alternative method, the handle does not need to be removed from the introducer in order to pass the endotracheal tube over the introducer. The endotracheal tube is advanced into a patient as discussed above, with the exception of removing the handle from the introducer.

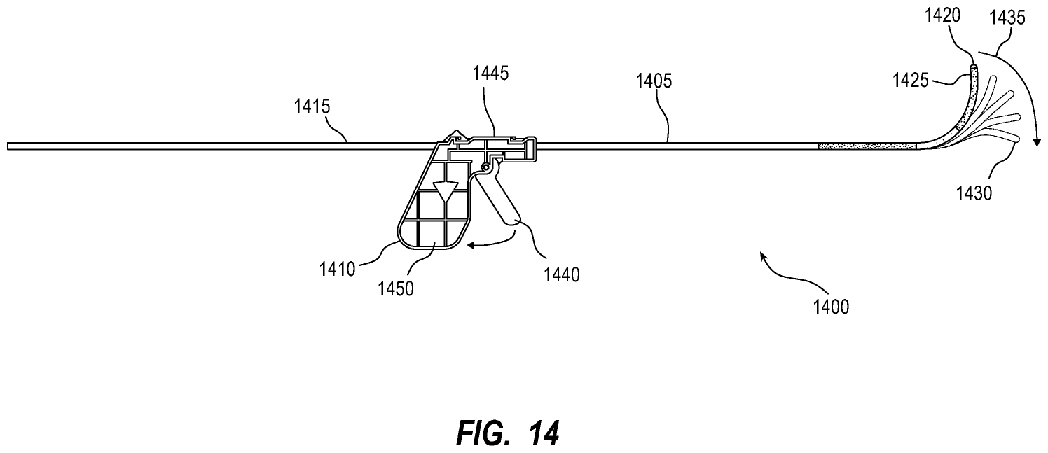

FIG. 14 is a side elevation view of a device 1400 that includes an introducer 1405 and a handle 1410. The device 1400 is configured with a dynamically shaping tip that is configured to articulate or move based on receiving an input. The device 1400 may also include indicators configured to enable a qualitative assessment of the depth of the introducer 1405 when it is positioned in a body of a patient. The introducer 1405 may be an example of the introducers described herein. The handle 1410 may be an example of the handles described above.

The introducer 1405 may be configured to be inserted into a patient in order to place a tube inside of a patient. For example, the introducer may be configured to be inserted into the nose or mouth of a patient and directed through the glottis of the patient and into the trachea of the patient. The introducer 1405 may include a shaft 1415 and a tip 1420. The shaft 1415 may be an example of the shaft 134 described herein. The tip 1420 may be an example of the tip 138 described herein.

In some examples, the tip 1420 is a dynamically shaping tip that is configured to move or articulate based on inputs received at the handle 1410. The tip 1420 may be positionable at a variety of positions. For example, the tip 1420 may articulate from a first position 1425 (e.g., resting position) to a second position 1430 (e.g., deployed position), or anywhere in-between based on the inputs received at the handle 1410. The tip 1420 may be configured to be in one of a plurality of intermediate positions 1435 based on the inputs received. The tip 1420 may include markings configured to provide qualitative assessment of the depth of the introducer within a patient.

In some cases, the first position 1425 (e.g., the resting position) may be a j-shaped curve. In some cases, the first position may be a straight shaft. In yet other cases, the first position 1425 may be any straight or curved position.

The tip 1420 may be configured to have one or more curved sections. For example, the first position 1425 may have a single curved section and the second position 1430 may have two curved sections, resulting in at least one inflection point of the curves defined by the tip 1420. The tip 1420 may include any number of curved section or any number of inflection points. For example, the tip 1420 may include one, two, three, four, five, six, seven, or eight curved sections and may include one, two, three, four, five, six, seven, or eight inflection points. In some cases, the tip 1420 may curve along a single plane, as shown in FIG. 14. In some cases, the tip 1420 may curve along multiple planes such that the tip 1420 curves in multiple directions.

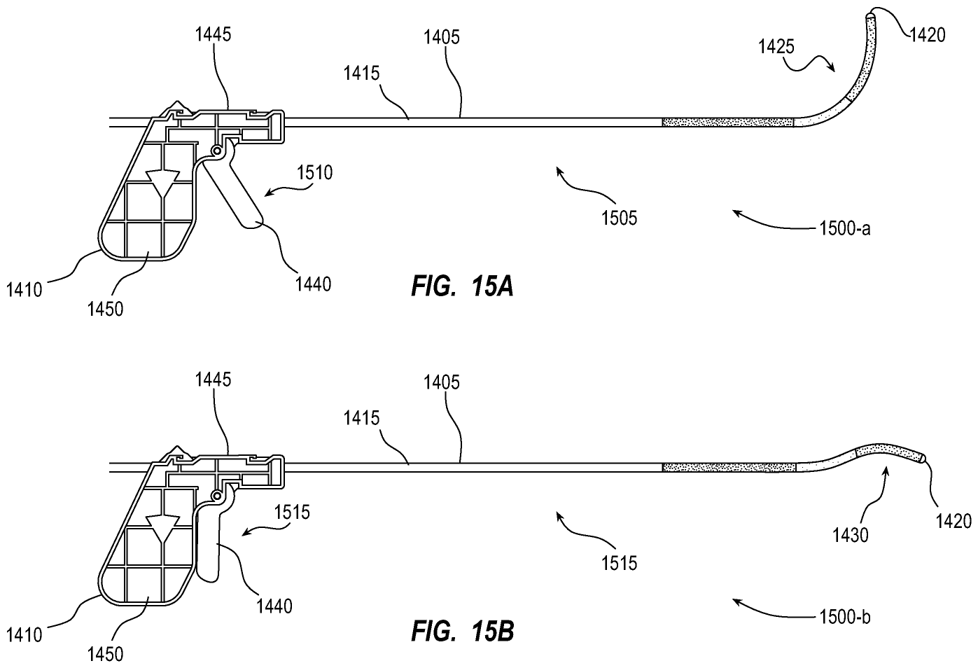

The handle 1410 may include a trigger 1440, a connection mechanism 1445, and a grip 1450. The connection mechanism 1445 is configured to couple with the introducer 1405 (e.g., the shaft 1415). In some cases, the connection mechanism 1445 may secure the introducer 1405 in a fixed position relative to the handle 1410. The handle 1410 may also include an actuation mechanism (not shown) that is configured to cause the tip 1420 to articulate, deform, or change shape in one or more directions. In some cases, the actuation mechanism is the connection mechanism 1445 or is a part of the connection mechanism 1445. The trigger 1440 may be coupled with the actuation mechanism and may be configured to cause the actuation mechanism to operate based on being actuated. The trigger 1440 may be any type of input devices that causes an action to occur based on receiving an input. Other examples of the trigger 1440 may include a button, a lever, joystick, touch screen, etc. As shown in FIGS. 15A and 15B, the trigger 1440 may be configured to be in a variety of positions based on the input received. For example, the trigger 1440 may be configured to be in a first position (e.g., a resting position) based on receiving no forces or input, a second position (e.g., a deployed position) based on receiving forces, or any position between the first position and the second position.

FIGS. 15A and 15B show side elevation views of a device 1500 (e.g., device 1500-a and device 1500-b) in a first position and in a second position. The device 1500 may be an example of the device 1400 described with reference to FIG. 14. As such, features that are named and/or numbered similarly may be embodied similarly.

FIG. 15A illustrates the device 1500-a in a first position 1505 (e.g., a resting position). In the first position 1505, the tip 1420 is in the first position 1425 (e.g., resting position) and the trigger 1440 is in a first position 1510 (e.g., a resting position). When in the first position 1505, a user may not be exerting any forces against trigger 1440 of the handle 1410.

FIG. 15B illustrates the device 1500-b in a second position 1515 (e.g., deployed position). In the second position 1515, the tip 1420 is in the second position 1430 (e.g., deployed position) and the trigger 1440 is in a second position 1520 (e.g., deployed position). When in the second position 1525, a user may be exerting a force against the trigger 1440 of the handle 1410. In some cases, the second position 1515 may be defined as the position when the trigger 1440 has reached its maximum movement position. In such situations, additional force applied in the same direction may not move the trigger 1440 any longer. For example, the second position 1515 may occur when movement of the trigger 1440 is arrested or stopped because the trigger 1440 is butting up against the grip 1450 of the handle 1410.

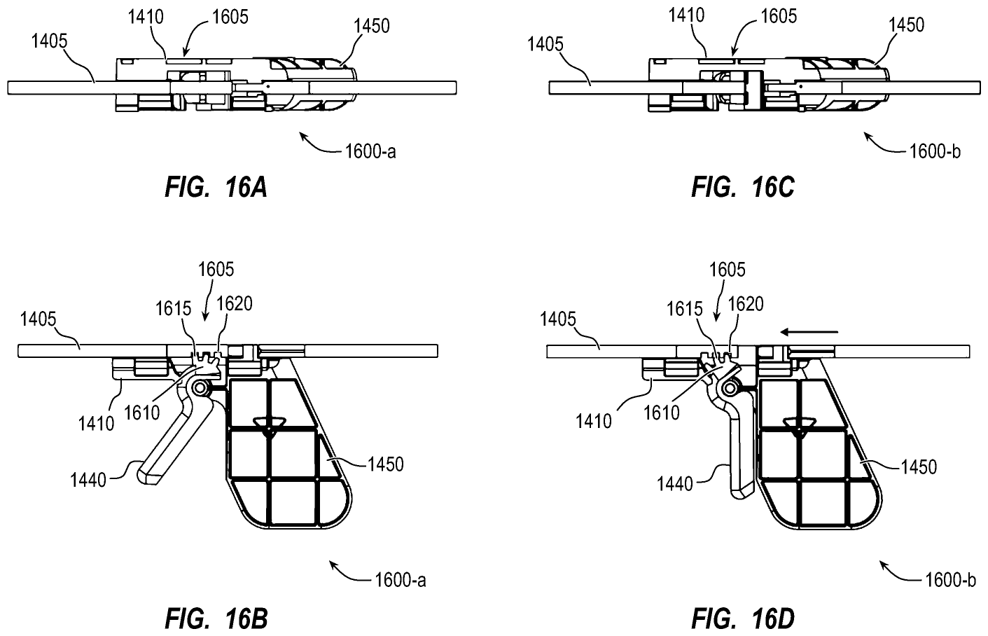

FIGS. 16A, 16B, 16C, and 16D views of a device 1600 (e.g., device 1600-a and device 1600-b) in a first position and in a second position. The device 1600 may be an example of the devices 1400 and 1500 described with reference to FIGS. 14, 15A, and 15B. As such, features that are named and/or numbered similarly may be embodied similarly. Devices 1600-a and 1600-b include an introducer 1405 and a handle 1410. The handle may include a trigger 1440 and a grip 1450, among other features.

Devices 1600-a and 1600-b illustrate an example of an actuation mechanism 1605 for causing the tip of the introducer 1405 to articulate, move, deform, or change shape. The actuation mechanism 1605 may be an example of gear actuation. The actuation mechanism 1605 may include a gear 1610 coupled with the trigger 1440. The gear 1610 may include a plurality of teeth 1615 that engage with a plurality of slots 1620 in the introducer 1405.

As a force is applied to the trigger 1440, the trigger 1440 may rotate, thereby causing the gear 1610 to rotate. When the gear 1610 rotates, the teeth 1615 exert a force against the slots 1620 of the introducer 1405, thereby causing the tip of the introduce to move or articulate, move, deform, or change shape. The device 1600-a illustrates a first position (e.g., a resting position). The device 1600-b illustrates a second position (e.g., a deployed position).

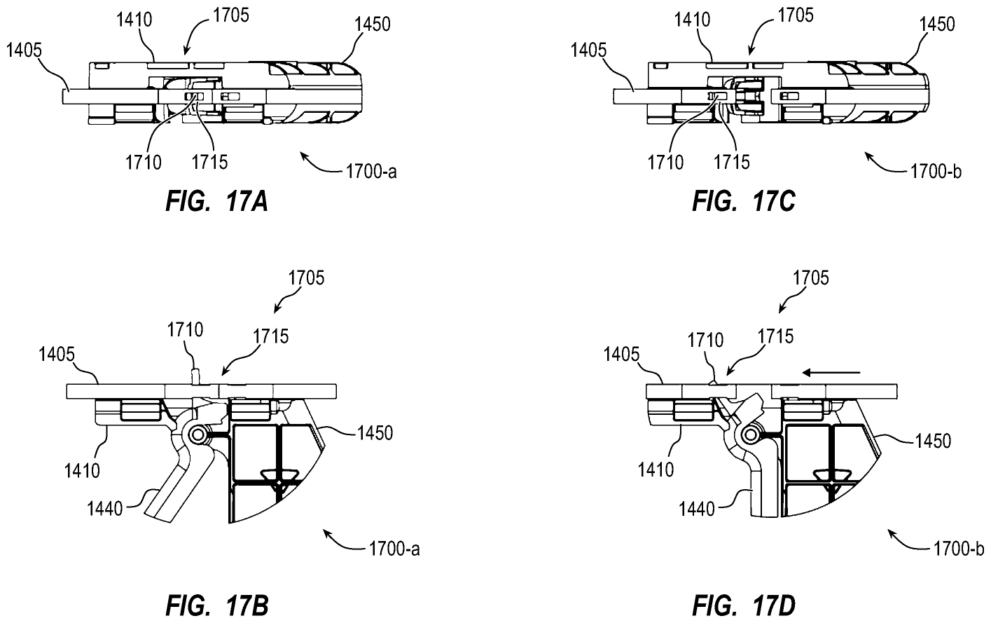

FIGS. 17A, 17B, 17C, and 17D views of a device 1700 (e.g., device 1700-a and device 1700-b) in a first position and in a second position. The device 1700 may be an example of the devices 1400 and 1500 described with reference to FIGS. 14, 15A, and 15B. As such, features that are named and/or numbered similarly may be embodied similarly. Devices 1700-a and 1700-b include an introducer 1405 and a handle 1410. The handle may include a trigger 1440 and a grip 1450, among other features.

Devices 1700-a and 1700-b illustrate an example of an actuation mechanism 1705 for causing the tip of the introducer 1405 to articulate, move, deform, or change shape. The actuation mechanism 1705 may be an example of pin and slot mechanism. The actuation mechanism 1705 may include a pin 1710 coupled with the trigger 1440. The pin 1710 may be inserted into a slot 1715 formed in the introducer 1405. A wall of the pin 1710 may engage with a wall of the slot 1715.

As a force is applied to the trigger 1440, the trigger 1440 may rotate, thereby causing the pin 1710 to move. When the pin 1710 moves, it may exert a force against the slot 1715 of the introducer 1405, thereby causing the tip of the introduce to articulate, move, deform, or change shape. The device 1700-a illustrates a first position (e.g., a resting position). The device 1700-b illustrates a second position (e.g., a deployed position).

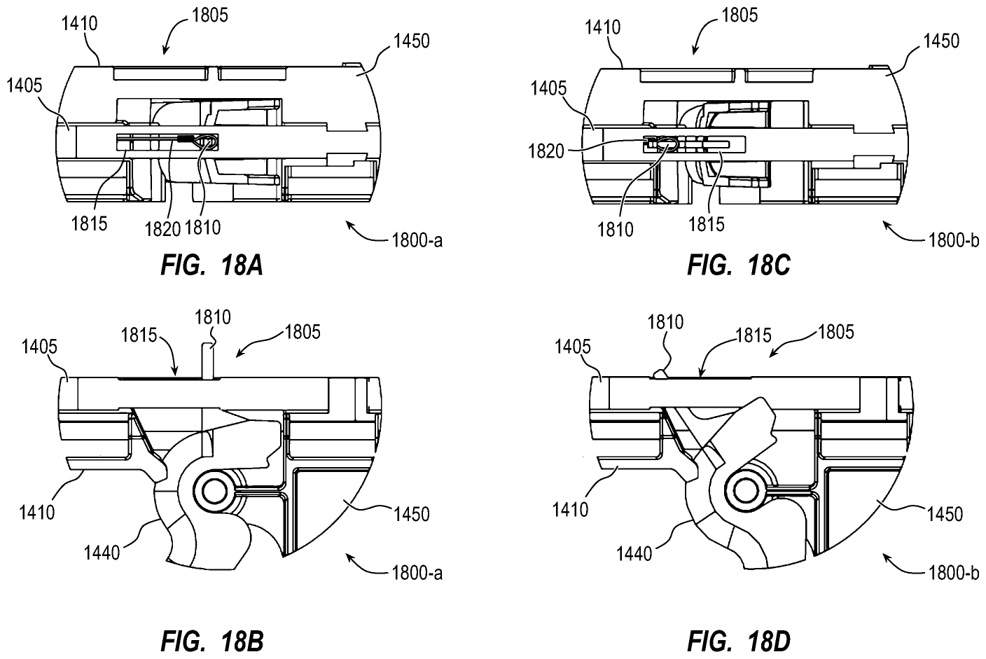

FIGS. 18A, 18B, 18C, and 18D views of a device 1800 (e.g., device 1800-a and device 1800-b) in a first position and in a second position. The device 1800 may be an example of the devices 1400 and 1500 described with reference to FIGS. 14, 15A, and 15B. As such, features that are named and/or numbered similarly may be embodied similarly. Devices 1800-a and 1800-b include an introducer 1405 and a handle 1410. The handle may include a trigger 1440 and a grip 1450, among other features.

Devices 1800-a and 1800-b illustrate an example of an actuation mechanism 1805 for causing the tip of the introducer 1405 to articulate, move, deform, or change shape. The actuation mechanism 1805 may be an example of an exposed wire mechanism. The actuation mechanism 1805 may include a pin 1810 coupled with the trigger 1440. The pin 1810 may be inserted into a slot 1815 formed in the introducer 1405. The slot 1815 of the introducer 1405 may expose a wire 1820 that is internal to the introducer 1405. The wire 1820 may be configured to cause the tip of the introducer 1405 to articulate, move, deform, or change shape. The wire 1820 may be couplable to the pin 1810. In some cases, the wire 1820 may include a loop that wraps around the pin 1810. In some cases, the wire 1820 may be inserted into the pin 1810.

As a force is applied to the trigger 1440, the trigger 1440 may rotate, thereby causing the pin 1810 to move. When the pin 1810 moves, it may exert a force against the wire 1820 of the introducer 1405, thereby causing the tip of the introduce to articulate, move, deform, or change shape. The device 1800-a illustrates a first position (e.g., a resting position). The device 1800-b illustrates a second position (e.g., a deployed position).

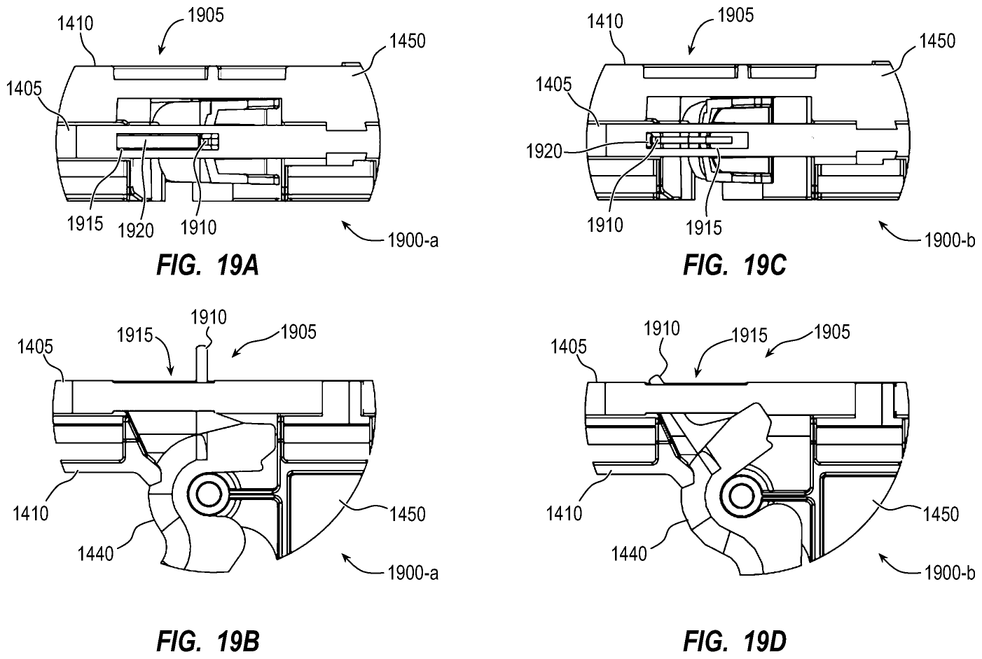

FIGS. 19A, 19B, 19C, and 19D views of a device 1900 (e.g., device 1900-a and device 1900-b) in a first position and in a second position. The device 1900 may be an example of the devices 1400 and 1500 described with reference to FIGS. 14, 15A, and 15B. As such, features that are named and/or numbered similarly may be embodied similarly. Devices 1900-a and 1900-b include an introducer 1405 and a handle 1410. The handle may include a trigger 1440 and a grip 1450, among other features.

Devices 1900-a and 1900-b illustrate an example of an actuation mechanism 1905 for causing the tip of the introducer 1405 to articulate, move, deform, or change shape. The actuation mechanism 1905 may be an example of a pushrod mechanism. The actuation mechanism 1905 may include a pin 1910 coupled with the trigger 1440. The pin 1910 may be inserted into a slot 1915 formed in the introducer 1405. The slot 1915 of the introducer 1405 may expose a pushrod 1920 that is internal to the introducer 1405. The pushrod 1920 may be configured to cause the tip of the introducer 1405 to articulate, move, deform, or change shape. The pushrod 1920 may be couplable to the pin 1910. In some cases, an end of the pushrod 1920 may engage with the pin 1910 or be moved by the pin 1910.

As a force is applied to the trigger 1440, the trigger 1440 may rotate, thereby causing the pin 1910 to move. When the pin 1910 moves, it may exert a force against the pushrod 1920 of the introducer 1405, thereby causing the tip of the introduce to articulate, move, deform, or change shape. The device 1900-a illustrates a first position (e.g., a resting position). The device 1900-b illustrates a second position (e.g., a deployed position).

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

What is claimed is:

1. An introducer for mounting an endotracheal tube, the introducer comprising:
a shaft comprising:
a proximal shaft portion; and
a distal tip portion extending from the distal shaft portion, wherein the distal top portion includes a qualitative depth assessment system to allow qualitative assessment of distal tip portion placement; and
a handle removably connected to the proximal shaft portion.

2. The introducer of claim 1, wherein the qualitative depth assessment system includes a plurality of depth assessment bands, each depth assessment band being visually distinct from an other depth assessment band.

3. The introducer of claim 2, wherein each depth assessment band has a visually distinct color or pattern from an other depth assessment band.

4. The introducer of claim 1, wherein the distal tip portion has a round shape and a closed end.

5. The introducer of claim 1, wherein the qualitative depth assessment system allows a user to evaluate tip placement based on an observation of the qualitative depth assessment system relative to an anatomical structure of a patient.

6. The introducer of claim 5, wherein the anatomical structure of the patient is a glottis of the patient.

7. The introducer of claim 5, wherein the anatomical structure of the patient is a lip of the patient.

8. The introducer of claim 5, wherein the anatomical structure of the patient is a tooth of the patient.

9. The introducer of claim 1, wherein the handle comprises a control mechanism, the control mechanism comprising a trigger.

10. The introducer of claim 9, wherein actuating the control mechanism causes the tip of the introducer to articulate, deform, or change shape.

11. The introducer of claim 10, wherein the tip of the introducer articulates, deforms, or changes shape away from or towards a longitudinal axis of the shaft.

12. The introducer of claim 1, in combination with an endotracheal tube, wherein the endotracheal tube is capable of being placed over and advanced along the introducer with the handle removed.

13. The introducer of claim 12, wherein the tip of the introducer extends from a distal end of the endotracheal tube.

14. The introducer of claim 1, wherein the shaft comprises a bulge extending between the proximal shaft portion and distal tip portion.

15. The introducer of claim 14, wherein the proximal shaft portion has a first diameter and the bulge has a second diameter, wherein the second diameter is larger than the first diameter.

16. The introducer of claim 1, wherein the introducer comprises a distal tip portion having a third diameter, wherein the third diameter is smaller than the first diameter, and wherein the third diameter tapers to the first diameter.

17. A method for inserting an endotracheal tube in a patient comprising:
inserting a blade of a laryngoscope in a mouth of the patient;
viewing a glottis of the patient with the laryngoscope;
inserting an introducer comprising a handle into the glottis of the patient;
removing the handle from the introducer;
inserting the endotracheal tube over the introducer and into the glottis of the patient; and
removing the introducer from the endotracheal tube while the endotracheal tube remains in the patient.

18. The method of claim 17, wherein the handle comprises a control mechanism, the control mechanism comprising a trigger, and wherein actuating the control mechanism causes the tip of the introducer to articulate, deform, or change shape.

19. The method of claim 17, wherein the introducer comprises a qualitative depth assessment system to allow qualitative assessment of introducer placement in the patient.

20. The method of claim 17, wherein the wherein the qualitative depth assessment system includes a plurality of depth assessment bands, each depth assessment band being visually distinct from an other depth assessment band.

21. The method of claim 20, wherein each depth assessment band has a visually distinct color or pattern from an other depth assessment band.

22. The method of claim 21, wherein the qualitative depth assessment system allows a user to evaluate tip placement based on an observation of the qualitative depth assessment system relative to an anatomical structure of a patient.

23. The method of claim 22, wherein the observation includes an observation of a depth assessment band of the depth assessment system relative to a structure within the glottis of the patient.

24. The method of claim 23, wherein the structure within the glottis of the patient is the glottis.

25. The method of claim 24, wherein the appropriate depth assessment band is adjacent to the glottis when a tip of the introducer is safely positioned in the glottis of the patient.

26. An introducer for mounting an endotracheal tube, the introducer comprising:
a shaft comprising:
a proximal shaft portion;
a distal shaft portion comprising a distal tip portion extending from the shaft portion, the shaft comprising a plurality of qualitative depth assessment bands, each depth assessment band having a visually distinct color or pattern from an other depth assessment band; and
a control wire at least partially disposed within both the distal shaft portion and the proximal shaft portion and configured to cause the tip portion of the proximal shaft portion to maintain a curved configuration.

27. The introducer of claim 26, further comprising a push rod at least partially disposed within both the distal shaft portion and the proximal shaft portion, the push rod configured to cause the tip portion of the proximal shaft portion to straighten when the push rod is extended in a distal direction.

28. The introducer of claim 27, wherein the push rod is made from a material that has a stiffness greater than a stiffness of the tip portion.

29. The introducer of claim 26, wherein the distal shaft portion and the proximal shaft portion comprise a continuous exterior surface.

30. The introducer of claim 26, wherein the shaft comprises a lumen, and the control wire is located within the lumen of the shaft and is moveable within the lumen.

31. The introducer of claim 26, wherein the control wire is fixedly connected to the tip of the tip portion.

32. The introducer of claim 26, wherein the shaft comprises a bulge extending between the proximal shaft portion and distal tip portion having a second diameter, wherein the second diameter is larger than the first diameter.

33. The introducer of claim 26, wherein the introducer comprises a distal tip portion having a second diameter, wherein the second diameter is smaller than the first diameter, and wherein the second diameter tapers to the first diameter.

34. The introducer of claim 26, wherein the tip portion has a round shape and a closed end.

35. An introducer for mounting an endotracheal tube, the introducer comprising:
a shaft comprising:
a proximal shaft portion; and
a distal tip portion extending from the shaft portion; wherein the distal tip portion may be articulated at more than one point along its length.

36. The introducer of claim 35, wherein the distal tip portion may be articulated to form a substantially j-shaped curve.

37. The introducer of claim 35, wherein the distal tip portion may be articulated to form a substantially s-shaped curve.

38. The introducer of claim 35, further comprising a qualitative depth assessment system to allow qualitative assessment of tip placement.

39. The introducer of claim 38, wherein the qualitative depth assessment system includes a plurality of depth assessment bands, each depth assessment band being visually distinct from an other depth assessment band.

40. The introducer of claim 39, wherein each depth assessment band has a visually distinct color or pattern from an other depth assessment band.

41. The introducer of claim 40, wherein the qualitative depth assessment system allows a user to evaluate tip placement based on an observation of the qualitative depth assessment system relative to an anatomical structure of a patient.

42. The introducer of claim 41, wherein the anatomical structure of the patient is a glottis of the patient.

43. The introducer of claim 41, wherein the anatomical structure of the patient is a lip of the patient.

44. The introducer of claim 41, wherein the anatomical structure of the patient is a tooth of the patient.

45. The introducer of claim 35, in combination with an endotracheal tube, wherein the introducer is loaded within the endotracheal tube.

46. The introducer of claim 45, wherein the tip of the introducer extends from a distal end of the endotracheal tube.

47. The introducer of claim 35, further comprising a bulge between the proximal shaft portion and distal tip portion.

48. The introducer of claim 47, wherein the proximal shaft portion has a first diameter and the bulge has a second diameter, wherein the second diameter is larger than the first diameter.

49. The introducer of claim 35, wherein the tip has a round shape and a closed end.
